(19) United States
(12) Patent Application Publication
(10) Pub. No.: US 2020/0352613 A1
(43) Pub. Date: Nov. 12, 2020

(54) TEMPLATE FOR FACILITATING THE INSTALLATION OF A BONE PLATE

(71) Applicant: SPINAL BALANCE, INC., Swanton, OH (US)

(72) Inventors: Faezah Agarwal, Swanton, OH (US); Aakash Agarwal, Swanton, OH (US)

(73) Assignee: SPINAL BALANCE, INC., Swanton, OH (US)

(21) Appl. No.: 16/765,896

(22) PCT Filed: Nov. 21, 2018
(86) PCT No.: PCT/US18/62188

§ 371 Date: May 21, 2020

Related U.S. Application Data
(60) Provisional application No. 62/590,127, filed on Nov. 22, 2017.

(51) Int. Cl. A61B 17/80; A61B 17/70

(52) U.S. Cl. CPC A61B 17/808; A61B 17/7059

(57) ABSTRACT

An assembly includes a template and a bone plate. The template includes abase, a retaining mechanism provided on the base, and a plurality of openings formed through the base. The bone plate includes a body having a plurality of openings formed through the body. The retaining mechanism releasably connects the base of the template to the body of the bone plate such that the plurality of openings formed through the base of the template is aligned with the plurality of openings formed through the body of the bone plate. A method of securing adjacent portions of a fractured bone or adjacent bones using the assembly is also disclosed.

### CROSS-REFERENCE TO RELATED APPLICATIONS

This application claims the benefit of U.S. Provisional Application No. 62/590,127, filed Nov. 22, 2017, the disclosure of which is incorporated herein by reference.

### BACKGROUND OF THE INVENTION

This invention relates in general to medical devices that are used in orthopedic surgeries. In particular, this invention relates to an improved structure for a template for facilitating the installation of a bone plate on adjacent portions of a fractured bone or between adjacent bones, such as a pair of vertebrae in a spinal column.

Bone fractures and other orthopedic injuries usually take a substantial time to heal, during which the bone is unable to support physiological loads. It is well understood that stabilization of a fractured bone may be accomplished using an implant, such as a bone plate, that is connected to adjacent portions of the fractured bone. Additionally, a bone plate may be connected to adjacent bones, such as a pair of vertebrae in a spinal column, to provide support therebetween when an intermediate disk is removed or repaired. A wide variety of bone plates and other medical devices are known and used for these purposes.

Typically, the bone plate is fastened to the bone or bones at multiple locations to immobilize a fracture or reconstructed area. To facilitate this, the bone plate is usually provided with a plurality of holes that permit the bone plate to be affixed to the bone or bones by respective surgical screws extending therethrough. Before a bone plate is affixed to the bone or bones, it is desirable that the bone plate preliminarily have a shape that conforms with the natural or desired reconstructed shape of the bone or bones. The characteristics of the shape of the bone plate surface may include the curvature and the height of the plate, as well as the orientation of the holes that define the trajectories of the surgical screws used to affix the bone plate to the bone or bones.

In the past, a template has been used to facilitate the shaping of the bone plate prior to installation on the bone or bones. Known templates are reusable devices that are initially positioned against the bone or bones, then manually reshaped to the desired shape of the bone plate using finger pressure. Although known templates are effective, substantial force is required to deform them into the desired shape for use with the bone plate, and thorough cleaning is required after every use to minimize the chance of infection. Furthermore, the deformation process makes the template more susceptible to hosting undesirable microorganisms or other foreign substances because of corrosion and crack development. Lastly, known bone plates have no features to facilitate the orientation of the bone screw trajectory, which is an important step for successful fixation of the bone plate to the bone or bones. Thus, it would be desirable to provide an improved structure for a template for facilitating the installation of a bone plate on adjacent portions of a fractured bone or between adjacent bones, such as a pair of vertebrae in a spinal column, that avoids these problems.

### SUMMARY OF THE INVENTION

This invention relates to an improved structure for a template for facilitating the installation of a bone plate on adjacent portions of a fractured bone or between adjacent bones, such as a pair of vertebrae in a spinal column. The bone plate template is pre-formed in various sizes, is made from an inexpensive and relatively flexible plastic or elastomeric material that mitigates the process of reshaping thereof, and assists in determining an ideal bone screw trajectory. The improved template of this invention comes in multiple sizes that are identical to the various bone plates available to the user at the time of surgery. The template may initially be attached to or detached from the bone plate, preferably inside a sterile package.

Various aspects of this invention will become apparent to those skilled in the art from the following detailed description of the preferred embodiments, when read in light of the accompanying drawings.

### BRIEF DESCRIPTION OF THE DRAWINGS

FIG. 1 is a front perspective view of a first embodiment of a template for facilitating the installation of a bone plate on adjacent portions of a fractured bone or between adjacent bones in accordance with this invention.

FIG. 2 is a rear elevational view of the template illustrated in FIG. 1.

FIG. 3 is an end perspective view of the template illustrated in FIGS. 1 and 2.

FIG. 4 is an exploded perspective view of the template illustrated in FIGS. 1 through 3 and a bone plate in accordance with this invention.

FIG. 5 is a perspective view of the template and the bone plate illustrated in FIG. 4 shown assembled.

FIG. 6 is a perspective view of the assembled template and bone plate illustrated in FIG. 5 shown positioned adjacent two vertebrae in a spinal column.

FIG. 7 is a perspective view similar to FIG. 6 showing four screws aligned for insertion through the assembled template and bone plate and into the two vertebrae.

FIG. 8 is an enlarged elevational view showing two of the four screws partially inserted through the assembled template and bone plate and into the two vertebrae.

FIG. 9 is an elevational view similar to FIG. 8 showing the four screws fully inserted through the assembled template and bone plate and into the two vertebrae.

FIG. 10 is a perspective view similar to FIG. 6 showing the template removed from the bone plate.

FIG. 11 is a perspective view similar to FIG. 6 of a second embodiment of a template for facilitating the installation of a bone plate on adjacent portions of a fractured bone or between adjacent bones in accordance with this invention.

FIG. 12 is an exploded perspective view showing the second embodiment of the template illustrated in FIG. 11 removed from the bone plate.

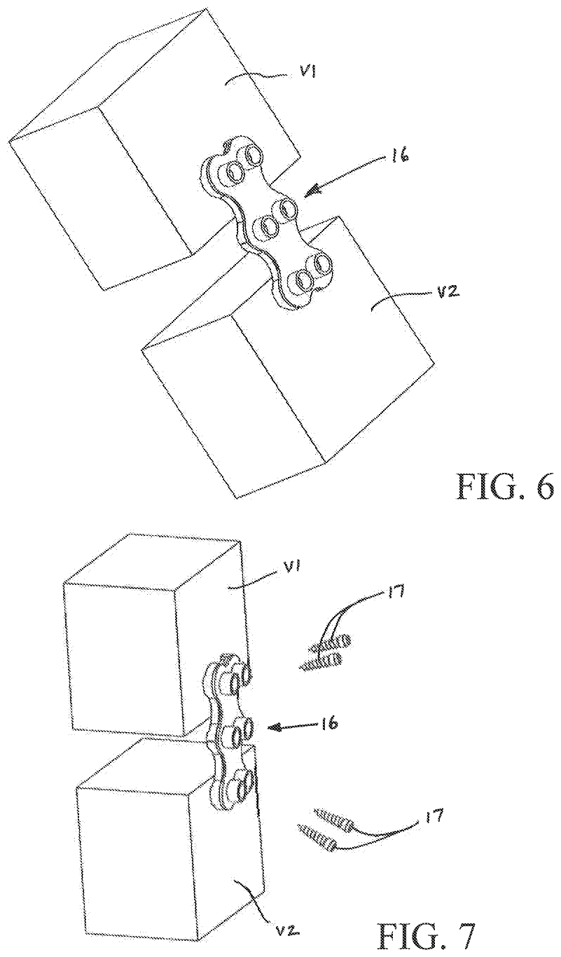

FIG. 11 is a perspective view similar to FIG. 6 of a third embodiment of a template for facilitating the installation of a bone plate on adjacent portions of a fractured bone or between adjacent bones in accordance with this invention.

FIG. 14 is a perspective view similar to FIG. 13 showing four screws aligned with insertion guides provided on the third embodiment of the template.

FIG. 15 is an exploded perspective view similar to FIG. 14 showing the third embodiment of the template and the insertion guides removed from the bone plate.

FIG. 16 is an exploded perspective view of a fourth embodiment of a template for facilitating the installation of a bone plate on adjacent portions of a fractured bone or between adjacent bones in accordance with this invention.

FIG. 17 is an exploded perspective view similar to FIG. 16 showing the fourth embodiment of the template assembled with the bone plate.

### DETAILED DESCRIPTION OF THE PREFERRED EMBODIMENTS

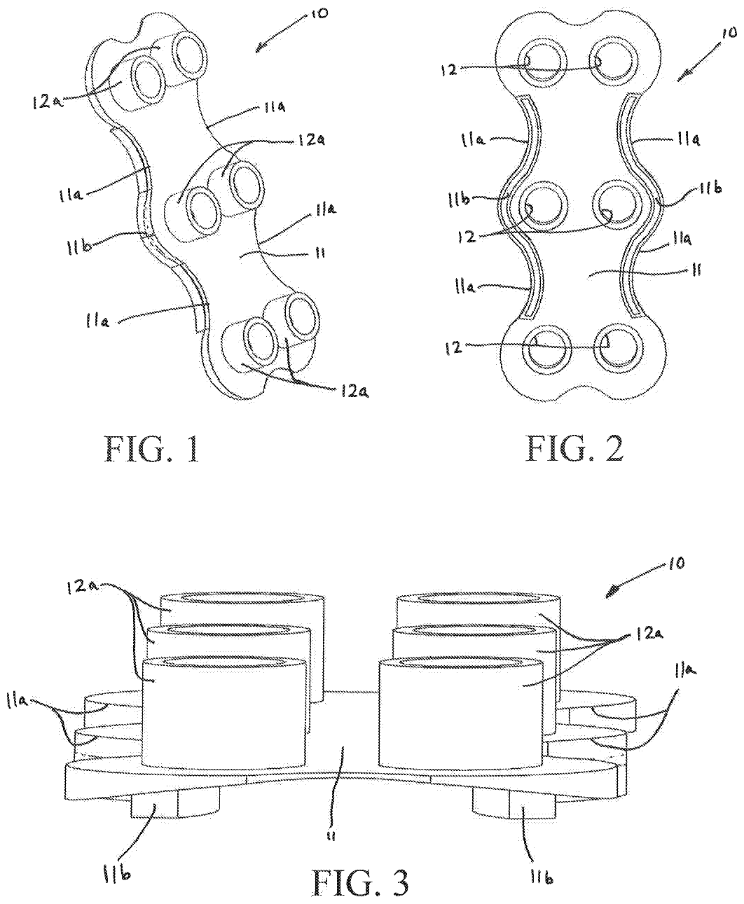

Referring now to the drawings, there is illustrated in FIGS. 1 through 3 a first embodiment of a template, indicated generally at 10, for facilitating the installation of a bone plate on adjacent portions of a fractured bone or between adjacent bones in accordance with this invention. The template 10 includes a base 11 that, in the illustrated embodiment, is generally flat and rectangular in shape, having a plurality of optional concavely-shaped curved edges 11a. However, the base 11 of the template 10 may have any desired shape. The base 11 of the template 10 includes a retaining mechanism. In the illustrated embodiment, this retaining mechanism is a pair of opposed walls 11b that extend generally perpendicularly from the base 11 of the template 10 adjacent to the curved edge portions 11a. However, any desired number of such walls 11b may extend in any desired direction or directions from any desired portions of the base 11. In the illustrated embodiment, each of the walls 11b is formed integrally with the base 11, although such is not required. The purpose of the walls 11b will be explained below. Preferably, the template 10 molded from a flexible plastic or elastomeric material. However, the template 10 may be manufactured using any desired process and from any desired material.

A plurality of openings 12 (see FIG. 2) is formed through the base 11 of the template 10. In the illustrated embodiment, the six of such openings 12 are provided in three pairs that are spaced across the length of the base 11. However, any desired number of such openings 12 may be provided in any desired orientation on the base 11. A hollow cylindrical guide 12a extends generally perpendicularly from the base 11 of the template 10 adjacent to each of the openings 12 in a direction that is opposite to the pair of walls 11b. However, any desired number of such hollow cylindrical guides 12a may extend in any desired direction from the base 11. In the illustrated embodiment, each of the hollow cylindrical guides 12a is formed integrally with the base 11, although such is not required. The purpose for the hollow cylindrical guides 12a will also be explained below.

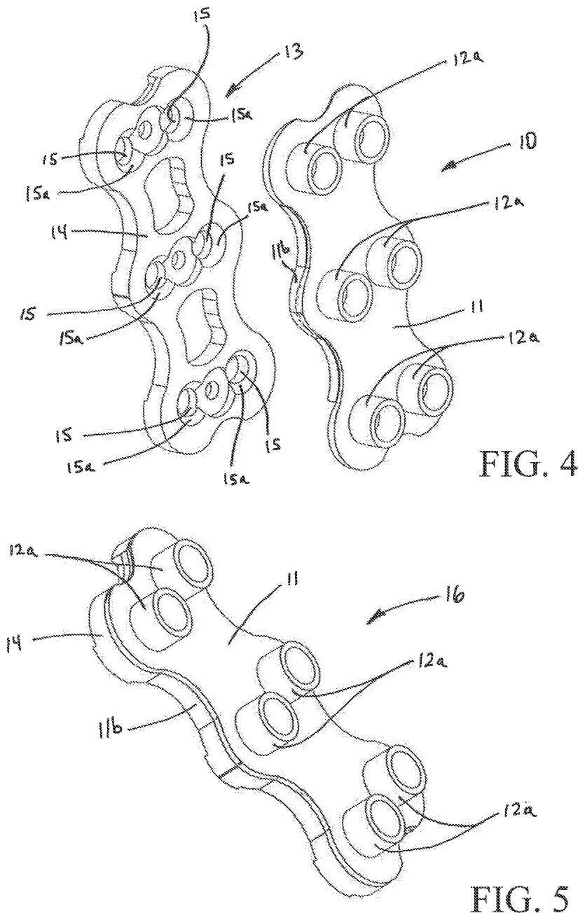

FIG. 4 illustrates a bone plate, indicated generally at 13, that can be assembled to and used with the template 10, as shown in FIG. 5. The bone plate 13 is, of itself, conventional in the art and is typically formed from a relatively rigid material, such as a metallic material. However, the bone plate 13 may be manufactured from any desired material or combination of materials. The illustrated bone plate 13 includes a body 14 that is generally flat and rectangular in shape and has a plurality of concavely-shaped curved edges 14a, although such is not required. For reasons that will become apparent below, the shape of the body 14 of the bone plate 13 preferably corresponds with the shape of the base 11 of the template 10. However, the body 14 of the bone plate 13 may have any desired shapes. A plurality of openings 15 is formed through the body 14 of the bone plate 13. Preferably, the number and locations of such openings 15 correspond with the number and locations of the openings 12 extending through the base 11 of the template 10, although such is not required. In the illustrated embodiment, a countersunk region 15a is provided in the surface of the body 14 of the bone plate 13 about each of the openings 15, although again such is not required.

As shown in FIG. 5, the template 10 is initially secured to the bone plate 13 to provide an assembly, indicated generally at 16, that can be used in the manner described below. Preferably, the template 10 is releasably secured to the bone plate 13 by a frictional or snap fit engagement of the walls 11b of the base 11 of the template 10 with corresponding edges of the body 14 of the bone plate 13. However, the template 10 may be secured to the bone plate 13 in any desired manner. When the template 10 is secured to the bone plate 13, the holes 12 formed through the base 11 of the template 10 are preferably aligned with the openings 15 formed through the body 14 of the bone plate 13. The purpose for releasably securing the template 10 to the bone plate 13 will be described below.

The manner of installation of the template 10 on the bone plate 13 will now be described. Initially a user, such as a surgeon, will grasp the outer surfaces of the sides of the template 10 with his or her fingers. The user can then move the body 14 of the template 10 into engagement with the base 10 of the bone plate 13, as shown FIGS. 4 and 5. As described above, the inner surfaces of the walls l lb of the base 11 of the template 10 engage the corresponding outer edges of the body 14 of the bone plate 13 in a frictional or snap fit relationship. Thus, the template 10 is releasably secured to the bone plate 13. If desired, the user may preliminarily pre-bend the template 10 and/or the bone plate 13 into a desired shape using any conventional methodology, such as described above. Such pre-bending may occur either before or after the template 10 is releasably secured to the bone plate 13.

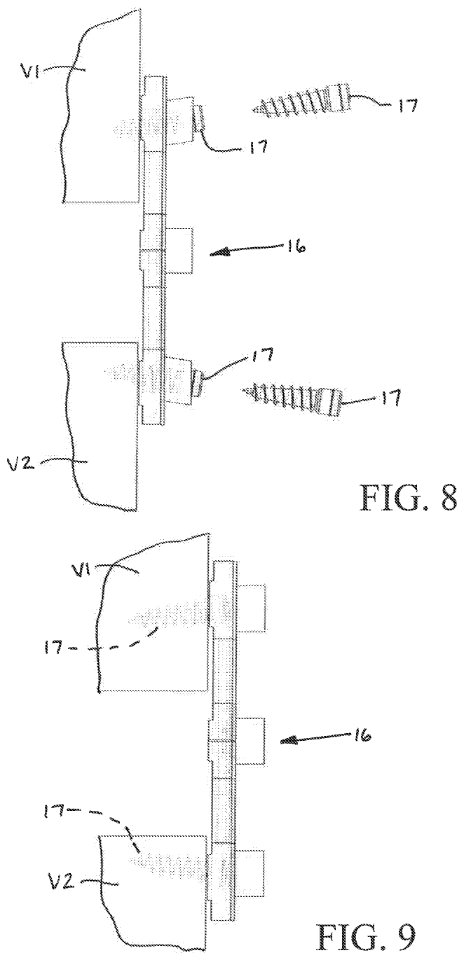

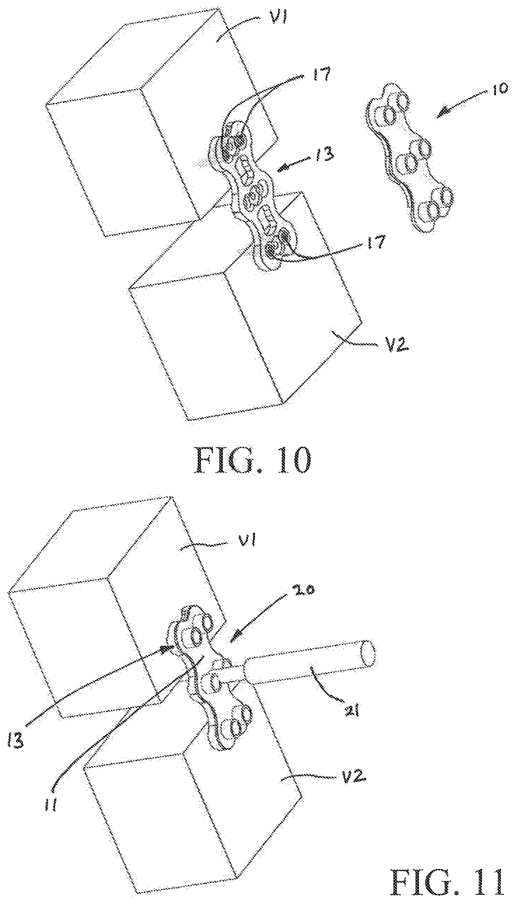

FIGS. 6 through 10 illustrate a method of installing the first embodiment of bone plate 13 on two adjacent vertebrae V1 and V2 in a spinal column. However, it will be appreciated that this invention may be used in connection with two portions of a single bone or between any two other types of bones. Initially, the assembly 16 of the template 10 and the bone plate 13 is positioned at a desired location relative to the vertebrae V1 and V2, as shown in FIG. 6. Any conventional method may be used to accomplish this initial positioning. Next, as shown in FIG. 7, a bone screw 17 is aligned with some or all of the aligned holes 12 formed through the base 11 of the template 10 and the openings 15 formed through the body 14 of the bone plate 13. Then, as shown in FIGS. 8 and 9, the bone screws 17 are advanced through the aligned holes 12 and the openings 15 into threaded engagement with the vertebrae V1 and V2. Any conventional tool may be used to advance the bone screws 17 in this manner. As a result, the assembly 16 of the template 10 and the bone 13 is secured to the vertebrae V1 and V2, as shown in FIG. 9. Lastly, the template 10 is removed from the bone plate 13, as shown in FIG. 10, leaving the bone plate 13 secured to the vertebrae V1 and V2 to provide stabilization therebetween. The surgeon or other user may use his or her fingers to grasp the template 10 and remove it from the bone plate 13.

Thus, the assembly 16 of the template 10 and the bone plate 13 is initially disposed adjacent to and installed on the vertebrae V1 and V2 using the bone screws 17, as described above. Subsequently, the template 10 is removed from the bone plate 13, leaving the bone plate 13 secured to the vertebrae V1 and V2 to provide stabilization therebetween. Importantly, throughout this entire process, the fingers of the surgeon or other user never contact the bone plate 13. Thus, a significant opportunity for contamination of the bone plate 13 is eliminated.

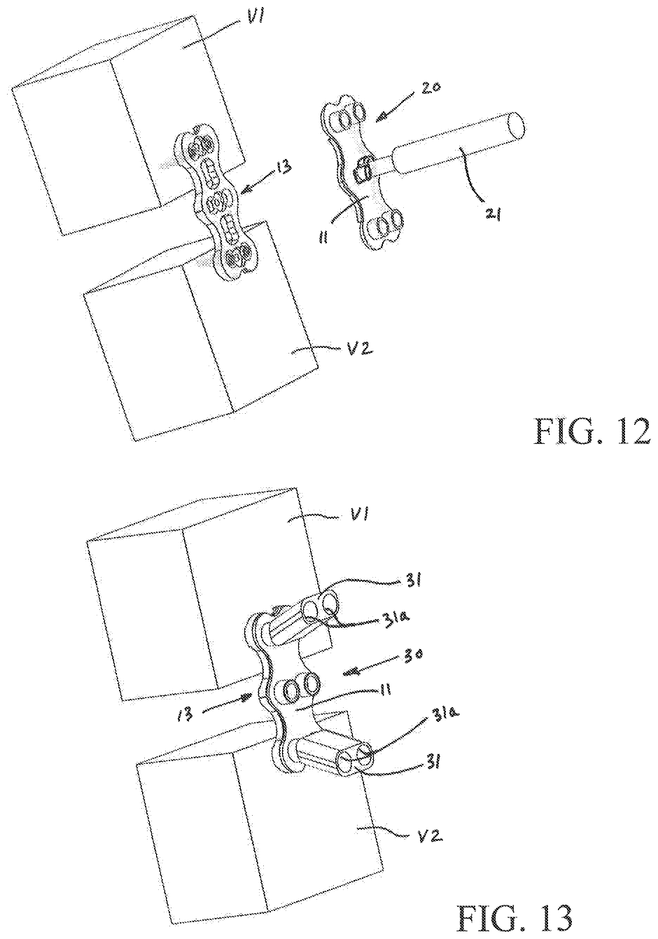

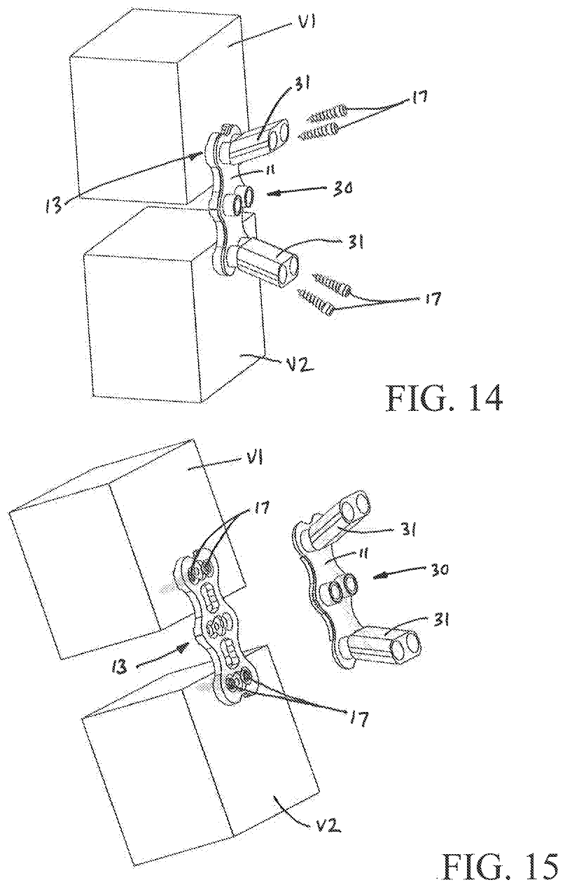

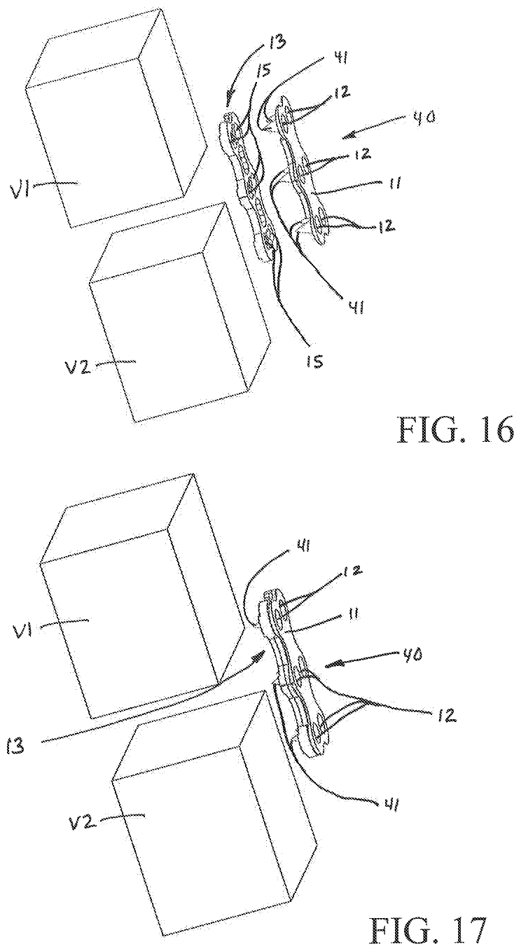

FIGS. 11 and 12 illustrate a second embodiment of a template, indicated generally at 20, for facilitating the installation of the bone plate 13 on adjacent portions of a fractured bone or between adjacent bones in accordance with this invention. The second embodiment of the template 20 is, in large measure, identical to the first embodiment of the template 10, and like reference numbers are used to indicate similar structures. However, the second embodiment of the template 20 further includes a handle 21 that is formed integrally with or otherwise connected to the base 11 of the template 20. In the illustrated embodiment, the handle 21 extends generally perpendicularly from the base 11 of the template 20. However, the handle 21 may extend any desired angle from the base 11. The handle 21 may be generally cylindrical in shape as shown, although such is not required. The handle 21 may be grasped by a surgeon or other user to facilitate the movement of the template 20 relative to the bone plate 13, and further to facilitate the movement of the assembly 16 of the template 20 and the bone plate 13 relative to the vertebrae V1 and V2, both in the manners described above.

FIGS. 13, 14, and 15 illustrate a third embodiment of a template, indicated generally at 30, for facilitating the installation of the bone plate 13 on adjacent portions of a fractured bone or between adjacent bones in accordance with this invention. The third embodiment of the template 30 is also, in large measure, identical to the first embodiment of the template 10, and like reference numbers are used to indicate similar structures. However, the third embodiment of the template 30 further includes one or more elongated guides 31 that are formed integrally with or otherwise secured to the base 11 of the template 30. In the illustrated embodiment, each of the guides 31 has two generally hollow and cylindrical openings 31a extending therethrough, and the inner ends of such openings 31a are aligned with two of the holes 12 formed through the base 11 of the template 30 through which the bone screws 17 will be inserted. However, a greater or lesser number of such guides 31 may be provided, and each of such guides 31 may have a greater or lesser number of such openings 31a. Preferably, each of the guides 31 is oriented at a non-perpendicular angle relative to the base 11 of the template 30 that corresponds with a desired angle at which the associated bone screw 17 is to be inserted into the respective vertebra V1 or V2. Depending upon the specific application, the guides 31 may be oriented at either the same angle or at differing angles relative to the base 11 of the template 30.

FIGS. 16 and 17 illustrate a fourth embodiment of a template, indicated generally at 40, for facilitating the installation of the bone plate 13 on adjacent portions of a fractured bone or between adjacent bones in accordance with this invention. The fourth embodiment of the template 40 is also, in large measure, identical to the first embodiment of the template 10, and like reference numbers are used to indicate similar structures. However, the fourth embodiment of the template 40 further includes one or more spikes 41 provided on the side of the base 11 that is adjacent to the bone plate 13. In the illustrated embodiment, a spike 41 is disposed adjacent to each of the openings 12 formed through the base 11 of the template 40, and each of the spikes 41 extends through a respective one of the openings 15 formed through the body 14 of the bone plate 13 when the template 40 is assembled onto the bone plate 13, as shown in FIG. 17. The spikes 41 may be formed having any desired structure or combination of structures, and any desired number of such spikes 41 may be provided on the template 40. The purpose of the spikes 41 is to positively position the assembly of the template 40 and the bone plate 13 on the vertebrae V1 and V2 prior to installing the bone screws 17 in the manner described above. After such bone screws 17 have been installed, the template 40 (including the spikes 41) is removed in the same manner described above.

As mentioned above, the bone plate 13 is preferably shaped to match the curvature and height of the vertebrae V1 and V2 before being attached thereto. This shape may suggest or dictate the orientation or trajectory of the bone screws 17 used to affix the bone plate 13 to the vertebrae V1 and V2. Using any one of the templates 10, 20, 30, and 40 for this purpose alleviates the need for the surgeon or other user to directly touch the bone plate 13, thus significantly reducing the chance of contamination and other problems. After the desired one of the templates 10, 20, 30, and 40 has been initially shaped in accordance with the vertebrae V1 and V2 to which it is to be secured, the bone plate 13 may then be shaped to fit the desired one of the templates 10, 20, 30, and 40, again minimizing the chance of contamination of the vertebrae V1 and V2 by the bone plate 13. Thus, limiting the amount of contact of the bone plate 13 with the vertebrae V1 and V2 significantly reduces the chances of infection, while reducing surgical time and handling of the bone plate 13.

For radiographic assessment, a radiographic material may be included in or on various areas of the templates 10, 20, 30, and 40 or the bone plate 13. The use of such radiographic material may assist in determining the proper position of the bone plate 13, the orientation of the bone screws 17, and like.

The principle and mode of operation of this invention have been explained and illustrated in its preferred embodiments. However, it must be understood that this invention may be practiced otherwise than as specifically explained and illustrated without departing from its spirit or scope.

What is claimed is:

1. A template for facilitating the installation of a bone plate on adjacent portions of a fractured bone or between adjacent bones, the template comprising:
a base;
a retaining mechanism provided on the base and adapted to releasably retain the base on a bone plate; and
a plurality of openings formed through the base and adapted to be aligned with corresponding openings formed through the bone plate.

2. The template defined in claim 1 wherein the retaining mechanism includes a pair of opposed walls extending from the base and adapted to engage opposed sides of the bone plate.

3. The template defined in claim 1 wherein a hollow guide extends from the template adjacent to each of the plurality of openings.

4. The template defined in claim 3 wherein each of the hollow guides is generally cylindrical in shape and extends generally perpendicularly from the template.

5. The template defined in claim 3 wherein each of the guides has two generally hollow and cylindrical openings extending therethrough.

6. The template defined in claim 1 further including a handle that extends from template.

7. The template defined in claim 1 wherein a spike is disposed adjacent to each of the openings.

8. An assembly of a template and a bone plate comprising:
a template including a base, a retaining mechanism provided on the base, and a plurality of openings formed through the base; and
a bone plate including a body having a plurality of openings formed through the body,
wherein the retaining mechanism releasably connects the base of the template to the body of the bone plate such that the plurality of openings formed through the base of the template is aligned with the plurality of openings formed through the body of the bone plate.

9. The assembly defined in claim 8 wherein the retaining mechanism includes a pair of opposed walls extending from the base that engage opposed sides of the bone plate.

10. The assembly defined in claim 8 wherein a hollow guide extends from the template adjacent to each of the plurality of openings.

11. The assembly defined in claim 10 wherein each of the hollow guides is generally cylindrical in shape and extends generally perpendicularly from the template.

12. The assembly defined in claim 10 wherein each of the guides has two generally hollow and cylindrical openings extending therethrough.

13. The assembly defined in claim 8 further including a handle that extends from template.

14. The assembly defined in claim 8 wherein a spike is disposed adjacent to each of the openings formed through the template and extends through the corresponding opening formed through the bone plate.

15. A method of securing adjacent portions of a fractured bone or adjacent bones comprising the steps of:
(a) providing a bone plate having a plurality of openings formed therethrough;
(b) providing a template having a plurality of openings formed therethrough;
(c) releasably securing the template to the bone plate to form an assembly wherein the plurality of openings formed through the bone plate is aligned with the plurality of openings formed through the template;
(d) positioning the assembly adjacent to adjacent portions of a fractured bone or adjacent bones;
(e) inserting fasteners through the aligned openings formed through the bone plate and the template to secure the bone plate to the adjacent portions of a fractured bone or adjacent bones; and
(f) removing the template from the bone plate.

16. The method defined in claim 15 including the step of shaping the template to fit a desired orientation of the adjacent portions of the fractured bone or adjacent bones prior to releasably securing the template to the bone plate.

17. The method defined in claim 15 wherein step (b) is performed by providing the template with a retaining mechanism including a pair of opposed walls, and wherein step (c) is performed by causing the opposed walls of the template to engage opposed sides of the bone plate to releasably secure the template to the bone plate.

18. The method defined in claim 15 wherein step (b) is performed by providing a template having a handle extending therefrom, and wherein step (d) is performed by using the handle to position the assembly adjacent to adjacent portions of a fractured bone or adjacent bones.

19. The method defined in claim 15 wherein step (b) is performed by providing the template with a spike disposed adjacent to each of the openings formed through the template, step (c) is performed by causing each spike to extend through the corresponding opening formed through the bone plate, and step (d) is performed by using the spikes to positively position the assembly adjacent to adjacent portions of a fractured bone or adjacent bones.
